(19) United States
(12) Patent Application Publication
(10) Pub. No.: US 2014/0363387 A1
(43) Pub. Date: Dec. 11, 2014

(54) COSMETIC COMPOSITIONS AND METHODS FOR ENHANCED UV PROTECTION

(71) Applicant: U.S. COSMETICS CORPORATION, DAYVILLE, CT (US)

(72) Inventors: Yoshiaki Kawasaki, Woodstock, CT (US); Michael Aaron Morin, North Grosvenordale, CT (US)

(21) Appl. No.: 14/467,150

(22) Filed: Aug. 25, 2014

Related U.S. Application Data
(62) Division of application No. 13/379,284, filed on Feb. 28, 2012, now abandoned.
(60) Provisional application No. 61/218,785, filed on Jun. 19, 2009.

(51) Int. Cl. A61K 8/02; A61K 8/35; A61K 8/26; A61K 8/37; A61Q 17/04; A61K 8/25

(52) U.S. Cl. CPC A61K 8/022; A61Q 17/04; A61K 8/25; A61K 8/26; A61K 8/37; A61K 8/35; A61K 2800/805
USPC 424/60; 424/59

(57) ABSTRACT

The present disclosure relates generally to cosmetic formulations having enhanced UV protection factors. The present disclosure relates particularly, but not by way of limitation, to UV-protecting cosmetic formulations comprising cosmetic powders and having low loadings of organic sunscreens.

### CROSS REFERENCE TO RELATED APPLICATIONS

This application is a divisional of U.S. application Ser. No. 13/379,284, filed Feb. 28, 2014, which is the U.S. National Stage of PCT/US2010/038966, filed Jun. 19, 2009 which in turn claims priority to U.S. Provisional Application No. 61/218,785, filed Jun. 19, 2009. The entire contents of all of the above related applications are incorporated herein by reference in their entireties.

### FIELD OF THE INVENTION

The present disclosure relates generally to cosmetic formulations having enhanced UV protection factors. The present disclosure relates particularly, but not by way of limitation, to UV-protecting cosmetic formulations comprising cosmetic powders and having low loadings of organic sunscreens.

### BACKGROUND

The information provided below is not admitted to be prior art to the present invention, but is provided solely to assist the understanding of the reader.

Many lotion-type sunscreens are currently available on the market. Typically, sunscreen formulas are water-in-oil (W/O) or oil-in-water (O/W) emulsions or are anhydrous systems. In order to obtain a high sun protection factor (SPF) and particularly a high protection factor relative to UV-A radiation (PFA), sunscreen formulations typically incorporate extensive amounts of oil-based, UV-active materials. The use of large amounts of oil-based, UV-actives causes the texture of the resulting sunscreens to be oily, greasy, tacky, and somewhat opaque. Oils are also undesirable because they may enhance the transdermal permeation of other formulation ingredients including ingredients for which transdermal administration may be inappropriate. In addition to these undesirable properties, the high loading of oil-based UV-actives often causes adverse skin reactions in sensitive individuals.

Commercial sunscreens are typically formulated to yield about 1 to 2 SPF units per weight percent (wt %) UV-active ingredient. For example, typical SPF 20 sunscreen formulations contain approximately 13% UV-active materials. It is often desirable to formulate sunscreen with much higher SPF ratings. To formulate sunscreens at the higher SPF rating requires corresponding increases in the concentration of oil-based, organic UV-actives.

It is desirable to formulate sunscreens with increasingly high SPF values to confer higher levels of protection. However, the current formulation metric implies higher degrees of unwanted side-effects. If organic UV absorbers are used in formulations at the lowest possible level, tactile issues and safety concerns would be ameliorated. Also, production costs would be lower as lesser amounts of raw materials are used in the formulation. Therefore, there exists a need to formulate sunscreens having lower amounts of organic UV-actives.

Other objects and advantages will become apparent from the following disclosure.

### SUMMARY OF INVENTION

The present disclosure relates to a cosmetic formulation comprising at least one organic, UV-active material and at least one cosmetic powder material such that the formulation has an SPF Index of at least 3.0. According to aspects of the disclosure, the cosmetic formulation has an SPF Index of at least 4.0. According to aspects of the disclosure, the cosmetic formulation has an SPF Index of at least 6.0. According to aspects of the disclosure, the cosmetic formulation has an SPF Index of at least 8.0. According to aspects of the disclosure, the cosmetic formulation has an SPF Index of at least 10.0.

According to aspects of the disclosure, the cosmetic formulation comprises at least one organic, UV-active. According to aspects of the disclosure, the organic UV-active is any organic sunscreen which absorbs, blocks, or otherwise mitigates ultraviolet radiation.

According to aspects of the disclosure, the cosmetic formulation comprises cosmetic powder from about 0.5 wt % to about 35 wt %. According to aspects of the disclosure, the cosmetic powder is selected from the group consisting of silicates, surface modified silicates, organic polymers, and mixtures thereof.

According to aspects of the disclosure, silicates may include hydrated silicates and may include materials, such as but not limited to: silica, mica, talc, sericite, kaolin, and mixtures thereof.

According to aspects of the disclosure, at least a portion of the cosmetic powder is a “substrate” of the covalent, surface-modifying reactions of the present disclosure. According to aspects of the disclosure, at least a portion of the cosmetic powder is a “filler” wherein the covalent, surface-modifying reactions of the present disclosure are not applied.

According to aspects of the disclosure, the silicates may be modified by having organic materials bonded onto a surface thereof. Non-limiting examples of surface-modified silicates include:
silica coated with triethoxycaprylylsilane, aluminum myristate, and disodium stearoyl glutamate;
talc coated with triethoxycaprylylsilane, aluminum myristate, and disodium stearoyl glutamate;
kaolin coated with triethoxycaprylylsilane, aluminum myristate, and disodium stearoyl glutamate;
Aluminum calcium sodium silicate coated with triethoxycaprylylsilane, aluminum myristate, and disodium stearoyl glutamate;
Mica coated with triethoxycaprylylsilane, aluminum myristate, and disodium stearoyl glutamate;
Titanated micas such as Flamenco Velvet, which is a pearl pigment, i.e., coated with triethoxycaprylylsilane, aluminum myristate, and disodium stearoyl glutamate.

According to an aspect of the disclosure, at least one hydroxyl of a cosmetic powder substrate may be covalently linked through a polyvalent metal to at least one organic compound comprising at least organic acid or acyl moiety. According to an aspect of the disclosure, at least one organic compound may bind or absorb at least a portion of an organic UV-active species.

According to an aspect of the disclosure, a cosmetic powder substrate may be bound to a surface-treatment agent comprising a complex of triethoxycaprylylsilane, aluminum myristate, and disodium stearoyl glutamate. According to aspects of the disclosure, the cosmetic formulation may further comprise at least one inorganic UV-active material. According to an aspect of the disclosure, the triethoxycaprylylsilane complex may bind or absorb at least a portion of an organic UV-active species.

According to aspects of the disclosure, non-limiting inorganic UV-active materials include titanium dioxide and zinc oxide. As used herein, the term “inorganic UV-active material” refers to a particulate, such as particulate titanium dioxide or zinc oxide. As is understood by persons of skill in the art, the titanium dioxide coating of a titanated mica is not UV-active.

According to aspects of the disclosure, the cosmetic formulation may optionally comprise a color pigment. Non-limiting, optional color pigments may include an iron oxide, such as a red, yellow, or black iron oxide. The color pigment may be a submicron particle, preferably from about 0.2 to about 0.3 micron.

According to certain aspects, the disclosed formulations may contain an emulsifying agent. According to aspects, the emulsifying agent may be particularly suitable for stabilizing mineral substances.

According to aspects of the disclosure, the cosmetic formulation may further comprise cosmetically-approved emollients (oil, waxes, etc.), humectants (polyols such as glycerin, butylene glycol and pentylene glycol), polysaccharides, and amino acids, preservatives, fragrances, and other additives typically used in cosmetic formulations.

According to aspects of the disclosure, the cosmetic formulations are substantially visually transparent.

Still other aspects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

FIG. 1 is a schematic representation of the reaction of a mono-carboxylate, surface-treatment agent with a cosmetic powder substrate; and,

FIG. 2 is a schematic representation of the reaction of a di-carboxylate, surface-treatment agent with a cosmetic powder substrate.

### DETAILED DESCRIPTION OF A PREFERRED EMBODIMENT

Co-pending U.S. patent application Ser. No. 11/142,468, assigned to the assignee of the present application, discloses a coated powder material containing a powder material having a surface layer that has been chemically immobilized with one or more surface-active agents and coated with an oil. Co-pending application Ser. No. 11/142,468 further discloses methods of making the coated powders. The present application incorporates by reference the entire content of application Ser. No. 11/142,468. The disclosed coated powder materials are dustless powders suitable for incorporation in various cosmetic and toiletry products.

Co-pending U.S. patent application Ser. No. 12/273,495, assigned to the assignee of the present application, discloses a water based slurry compositions, and methods for preparing water based slurry compositions. The disclosed water based slurry composition includes one or more pigments and a substrate, wherein the pigment or substrate has a surface that has been chemically immobilized with at least one surface-treatment agent (e.g., hydrophobic or hydrophilic); wherein the pigment adheres to the substrate, and wherein the pigment and substrate are dispersed in a water medium. The disclosed water based slurry composition also includes one or more pigments and a substrate, wherein the pigment or substrate has a surface that has been chemically immobilized with at least two surface-treatment agents (e.g., hydrophobic or hydrophilic); wherein the pigment adheres to the substrate, and wherein the pigment and substrate are dispersed in a water medium. A method for preparing a water based slurry composition includes providing at least one pigment and a substrate; contacting the substrate or pigment with a surface-treatment agent to produce a surface-modified substrate or pigment material, thereby producing a substrate having adhered thereto the pigment; blending the material until it is fully or partially extended, and dispersing the blended material in a liquid water based (aqueous) medium. The disclosed compositions are suitable for cosmetic applications. The present application incorporates by reference the entire content of application Ser. No. 12/273,495.

U.S. Pat. No. 6,482,441, assigned to Miyoshi Kasei, Inc., discloses surface-treated powders, suitable for cosmetic purposes. The present application incorporates by reference the entire content of U.S. Pat. No. 6,482,441. U.S. Pat. No. 6,482,441 discloses that powder materials may have various functional properties, such as: adhesion, aesthetic feel (touch), covering power, coloring power, and optical absorption and scattering. The '441 patent further discloses such powder properties become more fully realized as the particles are dispersed as primary-sized particles; these properties are attenuated as the particles become flocculated or agglomerated.

U.S. Pat. No. 6,036,945, and a continuation thereof (U.S. Pat. No. 6,280,710) assigned to Shamrock Technologies, Inc., discloses the use of micron-sized particles to nucleate the crystallization of a wax which may contain a sunscreen active. Titanium dioxide and zinc oxide are disclosed as preferred nucleation agents. The '945 patent discloses that because titanium dioxide and zinc oxide are UV-active they additively enhance the SPF value of the resulting wax powders.

The present inventors have surprisingly discovered a synergistic UV-activity where a micron-sized, coated particle is further coated with an organic UV-active material. This synergistic UV-activity is manifested by particles comprised of non UV-active materials such as clay (kaolin), silica, and nylon. This synergy allows the manufacture of cosmetic formulations having high SPF values, but relatively low concentrations of organic UV-active ingredients.

Commercial sunscreens are typically formulated to yield about 1 to 2 SPF units per weight percent (wt %) UV-active ingredient. For example, a typical marketed SPF 50 sunscreen formulations contain a total of approximately 27% UV-active materials such as Octinoxate 7.5%, Oxybenzone 6.0%, Ococrylene 8.0% and Octisalate 5.0%. The present disclosure relates to the term “SPF Index.” SPF Index is herein defined as the numerical ratio of SPF to the concentration of organic UV-actives in weight percent (wt %).

Persons of skill in the art are familiar with several methods for determining SPF values. In-vivo SPF values may be determined in accordance with the procedure set forth in the Food and Drug Administration (FDA) Tentative Final Monograph of proposed rules for sunscreen testing published in the Federal Register, Docket No. 78N-0038, May 12, 1993, 58 FR 28194. In-vivo PFA values may be determined in accordance with the procedure set forth in Federal Register Vol 72, No. 165 pp. 49070-49122, 21 CFR Parts 347 and 352, Sunscreen Drug Products for Over-the-Counter Human Use; Proposed Amendment of Final Monograph; Proposed Rule. In vitro SPF values may be determined in accordance with the method of Diffy, B. L. and Robson, J. (1989) (“A new substrate to measure sunscreen protection factor throughout the ultraviolet spectrum,” 40 J. Soc. Cosmet. Chem. 127-133, 1989).

For purposes of the present invention, SPF values were determined as proportional to the inverse transmittance at a given wavelength. A layer of Transpore™ (3M, Minneapolis, USA) tape is placed in a single layer on clean approximately 2 mm thick quarts slides. Preferably, an area of at least 40 cm² is applied to enable measurement six, non-overlapping spots. A minimum of three test samples and at least one control sample was prepared for each sunscreen to be tested. Sample plates were exposed to 280-400 nm UV irradiation in an SPF-290™ Ultraviolet Transmittance Analyzer (Optometrics LLC, Ayer, Mass., USA). The Transpore™ layers were evenly coated with approximately 2 mg/cm² of the appropriate sample or vehicle control was applied to the plates using a sponge. The plates were weighed on an analytical balance and allowed to equilibrate for 15 minutes. The sample plates were exposed to UV irradiation as before. Irradiation took place at 6 randomly selected points. SPF values were calculated according to Equation I using software supplied by the manufacturer.

$$SPF = \frac{\int A ( λ ) E ( λ ) d λ}{\int A ( λ ) E ( λ ) / MPF ( λ ) d λ} ,$$

For Equation I, E(λ) is the solar irradiance spectrum at wavelength λ, A(λ) is the erythemal action spectrum at wavelength λ, and MPF(λ) is the monochromatic protection factor at wavelength λ. MPF is roughly the inverse of the transmittance at a given wavelength.

Organic sunscreens for use in the invention include any organic sunscreen which absorbs, blocks or otherwise mitigates ultraviolet radiation. Without wishing to limit the invention in any way, such sunscreen compositions include, but are not limited to, p-aminobenzoic acid, 2-ethoxyethyl-p-methoxy cinnamate, diethanolamine-p-methoxy cinnamate, digalloyl trioleate, 2,2-dihydroxy-4-methoxybenzophenone, ethyl-4-bis-(hydroxypropyl)aminobenzoate, 2-ethylhexyl-2-cyano-3,3-diphenyl acrylate, ethylhexyl-p-methoxy cinnamate, 2-ethylhexyl salicylate, glyceryl aminobenzoate, 3 3,5-trimethylcyclohexyl salicylate, lawsone with dihydroxyacetone, methyl anthranilate, 2-hydroxy-4-methoxy benzophenone, amyl-p-dimethylamino benzoate, 2-ethylhexyl-p-dimethylamino benzoate, 2-phenylbenzimidazole-5-sulphonic acid, red petroleum, 2-hydroxy-4-methoxybenzophenone-5-sulphonic acid, triethanolamine salicylate, Amiloxate, Ethylhexyl dimethoxybenzylidene dioxoimidazolidine propionate, Ethylhexyl methoxycrylene, and the like, and mixtures thereof.

In addition to the sunscreens recited above, suitable sunscreens for use in the inventive sunscreen compositions are set forth in Sunscreens Final Monogram, 64FR27666 (May 21, 1999). Sunscreens approved by the regulatory authorities of the U.S., EU, Australia, and Japan, and suitable for purposes of the present invention may include: PABA, octyldimethyl-PABA, Phenylbenzimidazole sulfonic acid, Cinoxate, Dioxybenzone (Benzophenone-8), Oxybenzone (Benzophenone-3), Homosalate, Menthyl anthranilate, Octocrylene, Octinoxate, Octisalate, Sulisobenzone, Trolamine salicylate, Avobenzone, Terephthalylidene Dicamphor Sulfonic Acid, 4-Methylbenzylidene camphor, Methylene Bis-Benzotriazolyl Tetramethylbutylphenol, Bis-ethylhexyloxyphenol methoxyphenol triazine, bisimidazylate, Drometrizole Trisiloxane, Sodium Dihydroxy Dimethoxy Disulfobenzophenone (Benzophenone-9), Octyl triazone, Diethylamino Hydroxybenzoyl Hexyl Benzoate, Iscotrizinol, Polysilicone-15, Amiloxate, Ethylhexyl Dimethoxybenzylidene Dioxoimidazolidine Propionate and mixtures thereof.

Non-limiting preferred sunscreens include: Octinoxate (ethylhexyl methoxycinnamate, Oxybenzone (benzophenone-3), mentyl anthranilate, octocrylene, homosalate, octisalate, avobenzone, and mixtures thereof.

The disclosed compositions include at least one cosmetic powder material. For purposes of the present disclosure, the term “cosmetic powder” includes those powdered cosmetic raw materials suitable for use in cosmetic and toiletry products with the exception of UV-active, inorganic materials such as titanium dioxide and zinc oxide. Suitable powder materials are disclosed in co-pending Ser. No. 11/142,468.

Powdered cosmetic raw materials may include extenders, polymeric powders, pearl pigments, inorganic color pigments, inorganic white pigments, and organic pigments (lakes).

Extenders may include inorganic materials such as, but not limited to, talc silica, other silicates such as, mica, sericite, kaolin (clay), aluminum calcium sodium silicate, i.e., beadyl Beads™), calcium carbonate, magnesium carbonate, barium sulfate, aluminum oxide, zirconium oxide, ceramic powders, such as boron nitride, and metal powders such as aluminum. The kaolin may be a natural or calcined kaolin or may be a delaminated kaolin such as is sold by KaMin LLC. Typically, an extender material has a size of from about 1 to about 40 microns.

Polymeric powders may include powdered materials such as, but not limited to, nylon, polyethylene, polystyrene, polymethylmethacrylate (PMMA), wool, cellulose, silk, starch powder. Typically, a polymeric powder material has a size of from about 1 to about 40 microns.

Pearl pigments are typically composed of an extender material coated with an inorganic pigment. A typical pearl pigment is a titanated mica such as Timron Super Silver™ produced by Rona/EM Industries. A titanated mica is formed by depositing titanium dioxide onto a mica surface. Typically, a pearl pigment has a size of from about 10 to about 250 microns.

Inorganic color pigments may include, but are not limited to Iron oxides (red, yellow, black, brown), ultramarine blue, ultramarine pink, ultramarine violet, manganese violet, chromium oxide green, chromium hydroxide green, and carbon black. In order to maximize color intensity, inorganic color pigments typically have a submicron particle size.

Non-limiting inorganic white pigments include titanium dioxide and zinc oxide. Pigmentary grades of inorganic white pigments typically are about 0.3 microns and ultrafine grades are typically less than 0.1 microns.

Non-limiting organic pigments (lakes) may include Red 4, Red 7, Red 22, Red 27, Yellow 5, Yellow 6, Green 3, Blue 1, beta-carotene, carmine, and chlorophyll.

In an embodiment of the present disclosure, the cosmetic powder may be selected from the group consisting of silicates, organic polymers, and mixtures thereof. Silicates of the present invention may include hydrated silicates and may include, but are not limited to silica, mica, talc, sericite, kaolin, and mixtures thereof.

The silicates may have their surfaces modified by coating with various organic and/or inorganic compounds. Non-limiting examples of coatings include mixtures comprising one or more of triethoxycaprylylsilane, aluminum myristate, and disodium stearoyl glutamate.

Formulations of the present invention may include an emulsifying agent. Particularly suited emulsifying agents are those capable of stabilizing mineral additives. A suitable emulsifying agent is a hydroxyethylacrylate/sodium acryloyldimethyltaurate copolymer formulated with squalene and polysorbate 60. Such a material is traded under the name Simulgel™ NS. Suitable emulsifying agents include RM2051® (Dow Corning) a dimethicone based thickening and emulsifying polymer (INCI name: Sodium Polyacrylate (and) Dimethicone (and) Cyclopentasiloxane (and) Trideceth-6 (generic name for polyethylene glycol ethers of tridecyl alcohol having an average of six ethylene oxide units) (and) PEG/PPG 18/18 Dimethicone).

Other conventional additives typically employed in cosmetic powder compositions may be employed in conjunction with the present invention. Such additives include, but are not limited to one or more preservatives such as methyl paraben, butyl paraben, propyl paraben, phenoxyethanol, benzoic acid, imidazolidinyl urea and other conventional preservatives, antioxidants, emollients, plasticizers, surfactants water proofing additives, botanical extracts and fillers including polyethylene, magnesium carbonate, methylcellulose, mica and the like.

In an embodiment of the present invention, the components of the powder compositions are dry blended together using conventional powder blending apparatus and procedures.

The cosmetic powders may be coated with one or more surface-active layers. Coating of the cosmetic powders may be performed as disclosed in U.S. Pat. No. 6,482,441 or in co-pending Ser. No. 11/142,468 or co-pending Ser. No. 12/273,495.

In an embodiment, the surface-treating agent is a fatty acid or salt thereof according to Formula I:

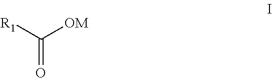

where R₁ may be an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group. Typically, but not by way of limitation, R₁ is a C₈ to C₂₄ carbon radical. M is a cation which may be independently selected from hydrogen, a metal or an organic base such as, but not limited to triethanolamine, aminomethyl propanol, or lysine.

In an embodiment, the surface-treating agent is an alkyl ether carboxylic acid or salt thereof according to Formula II:

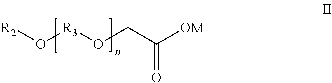

where R₂ may be an alkyl, alkylamide, alkenyl, alkynyl, alkoxy, aryl, cycloalkyl, arylalkyl group, all of which may be substituted by one or more hydroxy group, and may further be substituted by one or more alkoxyl, carboxyl, or oxo group. Typically, but not by way of limitation, R₁ is a C₈ to C₂₄ carbon radical. Spacer R₃ is typically ethylene, propylene, or butylene and typically the number, n of such spacer groups may vary from 0 to about 20. M is a cation which may be independently selected from hydrogen, a metal or an organic base such as, but not limited to triethanolamine, aminomethyl propanol, or lysine.

In an embodiment, the surface-treating agent is an acylamino acid or salt thereof according to Formula III:

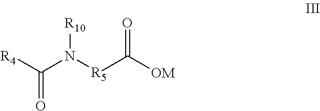

where R₄ and R₅ may each independently be an alkyl, alkylamide, alkenyl, alkynyl, alkoxy, aryl, cycloalkyl, arylalkyl group, any of which may be substituted by one or more hydroxy group(s), and may further be substituted by one or more alkoxyl, carboxyl, or oxo group(s). Typically, but not by way of limitation, R₄ is a C₈ to C₂₄ carbon radical and R₁₀ may be hydrogen or methyl. M is a cation which may be independently selected from hydrogen, a metal or an organic base such as, but not limited to triethanolamine, aminomethyl propanol, or lysine.

In an embodiment, the surface-treating agent is a 2-pyrrolidinone-5-carboxylic acid or salt thereof according to Formula IV:

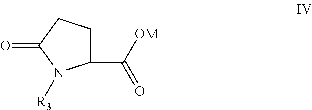

where R₃ may be an alkyl, alkylamide, alkenyl, alkynyl, alkoxy, aryl, cycloalkyl, arylalkyl group, any of which may be substituted by one or more hydroxy group, and may further be substituted by one or more alkoxyl, carboxyl, or oxo group(s). Typically, but not by way of limitation, R₄ is a C₈ to C₂₄ carbon radical. M is a cation which may be independently selected from hydrogen, a metal or an organic base such as, but not limited to triethanolamine, aminomethyl propanol, or lysine.

In an embodiment, the surface-treating agent is an acid polyamide or salt thereof according to Formula V:

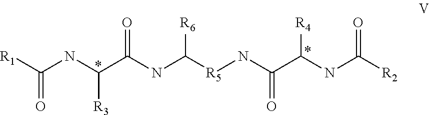

where each of R₁ and R₂ may be independently selected from hydrophobic alkyl, alkynyl, and alkenyl moieties. Typically, but not by way of limitation, each of R₁ and R₂ is independently a C₈ to C₂₄ carbon radical. Each of R₃ and R₄ may be independently selected from alkyl, alkynyl, and alkenyl amino acid moieties. Each of R₅ and R₆ may be independently selected from alkyl, alkynyl, and alkenyl moieties. At least one of R₃, R₄ and R₆ has a carboxylic group, which may be in an acid form (complexed with hydrogen) or may be in a salt form (complexed with a cation, which may be a metal or an organic base such as, but not limited to triethanolamine, aminomethyl propanol, or lysine).

In an embodiment, the surface-treating agent is a phospholipid or salt thereof, according to Formula VIa, or an alkyl ether phosphoric acid or salt thereof according to Formula VIb or Formula VIc:

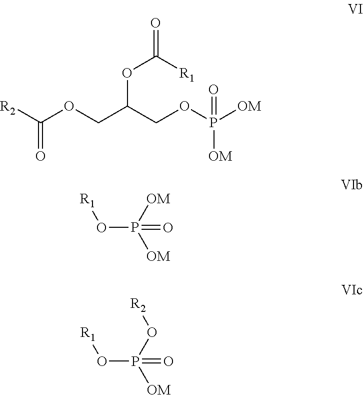

where each of R₁ and R₂ may be independently selected from alkyl, alkylamide, alkenyl, alkynyl, alkoxy, aryl, cycloalkyl, arylalkyl group(s), any of which may be substituted by one or more hydroxyl group(s), and may further be substituted by one or more alkoxyl, carboxyl, or oxo group(s). Typically, but not by way of limitation, each of R₁ and R₂ is independently a C₈ to C₂₄ carbon radical. Each M is a cation which may be independently selected from hydrogen, a metal, or an organic base such as, but not limited to triethanolamine, aminomethyl propanol, or lysine.

In an embodiment, the surface-treating agent is an amphoteric or salt thereof, according to Formula VIIa or VIIb:

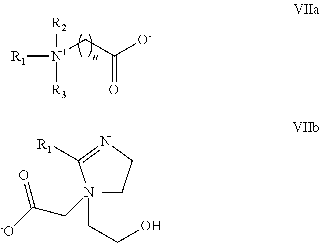

where each of R₁, R₂, and R₃ may be independently selected from alkyl, alkylamide, alkenyl, alkynyl, alkoxy, aryl, cycloalkyl, arylalkyl group(s), any of which may be substituted by one or more hydroxyl group(s), and may further be substituted by one or more alkoxyl, carboxyl, or oxo group(s). Typically, but not by way of limitation, R₁ is independently a C₈ to C₂₄ carbon radical.

In an embodiment, the surface-treating agent is a silane, according to Formula VIIIa-VIIIc:

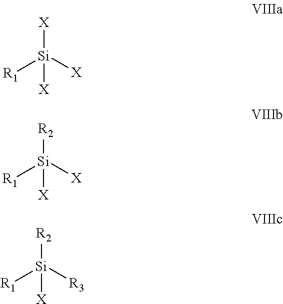

where each of R₁, R₂, and R₃ may be independently selected from alkyl, alkylamide, alkenyl, alkynyl, alkoxy, aryl, cycloalkyl, arylalkyl group(s), any of which may be substituted by one or more hydroxyl group(s), and may further be substituted by one or more alkoxyl, carboxyl, or oxo group(s). Typically, but not by way of limitation, R₁ is independently a C₈ to C₂₄ carbon radical. X is an alkoxy group such as, but not limited to methoxy, ethoxy, isopropoxy, isobutoxy or halogen (F, Cl, Br, or I).

In an embodiment, the surface-treating agent is a polysilane, according to Formula IX: [ ]

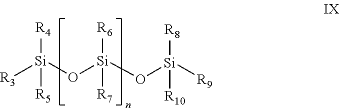

where each of R₃, R₄, R₅, R₆, R₇, R₈, R₉, and R₁₀ may be independently selected from alkyl, alkylamide, alkenyl, alkynyl, alkoxy, aryl, cycloalkyl, arylalkyl group(s), any of which may be substituted by one or more hydroxyl group(s), and may further be substituted by one or more halogen, alkoxyl, carboxyl, or oxo group(s). Index, n, may vary from 0 to about 60.

In an embodiment, the surface-treating agent is a tetrahydropyran-2-carboxylic acid or salt thereof, according to Formula X:

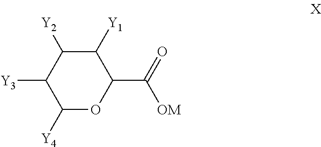

where each of Y₁, Y₂, Y₃, and Y₄ may be independently selected from a hydrogen, hydroxy, alkoxy, or oxo group. At least one of these is a hydroxy group. M is a cation which may be independently selected from hydrogen, a metal, or an organic base such as, but not limited to triethanolamine, aminomethyl propanol, or lysine.

In an embodiment, the surface-treating agent is a tetrahydrofuran-2-acetyl carboxylate, according to Formula XI:

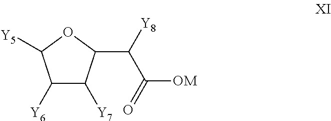

where each of Y₅, Y₆, Y₇, Y₈ is independently selected from hydrogen, hydroxy group, alkoxy group or oxo group and at least one of these is a hydroxy group. M is a cation which may be independently selected from hydrogen, a metal, or an organic base such as, but not limited to triethanolamine, aminomethyl propanol, or lysine.

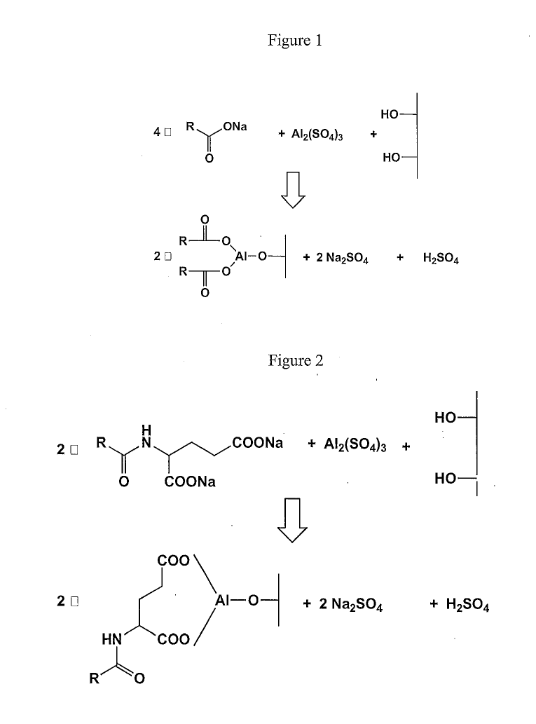

In an embodiment, at least one carboxyl moiety (—COOH) of a surface-treatment agent is covalently bridged to at least one hydroxyl moiety present on the surface of a substrate cosmetic powder. In an embodiment (FIG. 1), four equivalents of a mono-carboxylate, surface-treatment agent is mixed with a cosmetic powder substrate. The cosmetic powder is represented as a pair of hydroxyls. The carboxylates and hydroxyls are mixed with a polyvalent metal ion salt such as, but not limited to, aluminum sulfate (Al₂(SO₄)₃). The reaction proceeds such that two surface-treatment agent moieties are covalently crosslinked, through their carboxyl groups to a hydroxyl from the cosmetic powder surface. Four equivalents of a mono-carboxylate, surface-treatment agent yield two equivalents of a metal-bridged complex.

In an embodiment (FIG. 2), two equivalents of a dicarboxylate, surface-treatment agent is mixed with a cosmetic powder substrate. The carboxylates and hydroxyls are mixed with a polyvalent metal ion salt such as, but not limited to, aluminum sulfate (Al₂(SO₄)₃). The reaction proceeds such that a single surface-treatment agent moiety is covalently crosslinked, through its two carboxyl groups to a hydroxyl from the cosmetic powder surface. Two equivalents of di-carboxylate surface-treatment agent yields one equivalents of a metal-bridged complex.

Formulae I-XI are drawn where the surface-treatment agents comprise carboxyl substituents. In some embodiments of the present disclosure, some or all of the carboxyl groups may be replaced by sulfate groups (—SO₄) and/or phosphate groups (—PO₄).

The present disclosure provides general procedures for the surface treatment of the cosmetic powder. In an embodiment, a cosmetic powder is combined in a vessel with an amount of water or a mixture of water and organic solvent like ethanol, isopropanol, etc. to form a slurry. The cosmetic powder may be a single material or may be a mixture of the various substrate materials disclosed herein. Surface-treatment agents, such as disclosed in paragraphs 0046-0059 are added to the slurry and mixed. At least one polyvalent metal salt such as, but not limited to, aluminum sulfate, aluminum chloride, magnesium sulphate, magnesium chloride, calcium chloride, calcium sulfate, is added to immobilize the treating agents to the powder surface.

A liquid organic UV-active material is added to the slurry above. The UV-active may be a single substance or may be a combination of substances. Where the UV-active is a room-temperature solid, such as oxybenzone or avobenzone or a similar material, the solid should be dissolved in a liquid organic solvent. Preferably, the organic solvent should, itself, be a UV-active material such as, but not limited to, octocrylene or octinoxate.

In an embodiment, the UV-active material may be added to the cosmetic powder prior to the addition of the polyvalent metal salt. In an embodiment, the UV-active material may be added to the cosmetic powder after the addition of the polyvalent metal salt. In an embodiment, the UV-active material may be added to the cosmetic powder simultaneously with the addition of the polyvalent metal salt.

The covalent surface-treating reaction is allowed to proceed to completion and a “wetcake,” comprising a surface-treated cosmetic powder with bound UV-active, is separated from un-reacted materials. The separation may be effected by means including, but not limited to centrifugation and filtration.

The wet cake may be dispersed into water by mixing with a homogenizer, disperser, propeller mixer, or other mechanical means as is known in the art. Additional cosmetic materials are to be added to stabilize the system and to adjust the texture as a sunscreen formula. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

The “wetcake” residue resulting from filtration or centrifugation comprises a hydrophobic, surface-modified cosmetic powder dampened with residual water. The hydrophobic coating causes the UV-active sunscreen oils to adhere to the surface-modified cosmetic powder. The treatment acts as a surfactant layer between water and the oils and the cosmetic powder.

According to an embodiment, the wetcake may be at least partially dried. Drying may be at a temperature from about 105° C. to about 120° C. from about 1 to about 10 hours. Drying my be continued until a desired degree of dryness is obtained.

Alternatively, the various ingredients may be blended by “kneading.” The knead-blend method allows for the use of substantially less water than is required to form a wetcake. Knead-blending also produces a much drier product (knead-blend) compared to a wetcake.

A knead-blend may be formed by moistening a cosmetic powder with a solvent. The cosmetic powder may be a single substance or may be a mixture of several substances. The solvent may be water or a mixture of water and an organic solvent. The organic solvent is preferably a lower alcohol such as, but not limited to, ethanol or isopropanol. The organic solvent may be a mixture of solvents.

A surface-treatment agent, or a mixture thereof, is added to the moistened cosmetic powder and blended. Suitable surface-treatment agents are disclosed in paragraphs 0046-0059.

At least one polyvalent metal salt such as, but not limited to, aluminum sulfate, aluminum chloride, magnesium sulphate, magnesium chloride, calcium chloride, calcium sulfate, is added to immobilize the treating agents to the powder surface.

A liquid organic UV-active material is added to the slurry above. The UV-active may be a single substance or may be a combination of substances. Where the UV-active is a room-temperature solid, such as oxybenzone or avobenzone or a similar material, the solid should be dissolved in a liquid organic solvent. Preferably, the organic solvent should, itself, be a UV-active material such as, but not limited to, octocrylene or octinoxate.

The knead-blend, comprised of surface-treated cosmetic powder with bound UV-active, is retrieved as a moist composition.

In an embodiment, the UV-active material may be added to the cosmetic powder prior to the addition of the polyvalent metal salt. In an embodiment, the UV-active material may be added to the cosmetic powder after the addition of the polyvalent metal salt. In an embodiment, the UV-active material may be added to the cosmetic powder simultaneously with the addition of the polyvalent metal salt.

In an embodiment, the knead-blend may be at least partially dried. Drying may be at a temperature of from about 105° C. to about 120° C. for from about 1 to about 10 hours. Drying my be continued until a desired degree of dryness is obtained.

In an embodiment the dried surface-treated material with bound UV-actives may be dispersed into cosmetic formulations such as anhydrous systems or water-in-oil emulsions. In an embodiment the dried surface-treated material with bound UV-actives may be formulated as a powder system such as, but not limited to a pressed foundation powder or a loose powder. Other cosmetic ingredients may be added as appropriate. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

The wetcake or the knead-blend produced by any of the above methods may be optionally oven dried. Drying typically is performed overnight at 105° C. This method removes most or all of the retained water. The “dried” product consists of treated cosmetic powder containing the sunscreen oils. The lipophilic nature of the surface coating and the sunscreen actives allow for a stable system.

In an embodiment, the optionally-dried wetcake and/or knead-blend may be emulsified to form a stable oil-in-water (OW) system. An amount of water is added to the optionally-dried wetcake and/or knead-blend and mixed with a homogenizer, dispersion blade, or prop. The choice of blending instrument may depend on the amount of energy required as is apparent to a person of skill in the art. A rheological modifier (thickening agent) is added with continued mixing. The rheological modifier may be Simulgel™ NS or Dow RM2051™. Addition of the rheological modifier produces a final product having a creamy lotion texture.

Without being bound by theory, the inventors believe that it is the quality of the dispersion, specifically the treated cosmetic powder combined with the UV active oils that produce a “blanket” of coverage upon application, which provides the basis for the enhanced SPF.

In an embodiment, the optionally-dried wetcake and/or knead-blend may be emulsified to form a stable water-in-oil (WO) system. The optionally-dried wetcake and/or knead-blend may be homogenized with typical oils, such as, but not limited to isononyl isononanoate (“ININ”), cyclomethicone (cyclopentadimethylsiloxane, “D5”), isododecane, and emulsifiers, such as, but not limited to ABIL WE09 (a blend of polyglyceryl-4 isostearate (and) cetyl PEG/PPG-10/1 dimethicone (and) hexyl laurate).

The wetcake or knead-blend may be formulated with other cosmetic ingredients. Such additional materials may include, but are not limited to: emulsifying agents, preservatives, antioxidants, emollients, plasticizers, surfactants, waterproofing agents, botanical extracts, dyes, colorants, scent agents, perfumes, and mixtures thereof.

The invention may be illustrated by the following non-limiting examples.

### Example 1

TABLE I

| | Comparative | | | |
|---|---|---|---|---|
| | Example 1 | Example 1 | Example 2 | Example 3 |
| Ingredient | Wt % | wt % | wt % | wt % |
| UV-Actives | | | | |
| Octinoxate | 3.0 | 1.3 | 3.0 | 2.9 |
| Oxybenzone | 3.0 | 1.3 | 3.0 | 2.9 |
| Cosmetic Powder | | | | |
| Uncoated silica | — | — | 1.2 | — |
| Uncoated talc | — | — | 9.8 | — |
| Coated silica | — | 1.2 | — | 1.2 |
| Coated talc | — | — | — | 9.8 |
| Remaining | | | | |
| Ingredients | | | | |
| Hydroxyethyl | 2.5 | 5.0 | 5.0 | 5.0 |
| Acrylate/Sodium | | | | |
| Acryloyldimethyl | | | | |
| Taurate opolymer | | | | |
| (and) Squalane | | | | |
| (and) Polysorbate 60 | | | | |
| (Simulgel ™ NS) | | | | |
| Deionized water | QS 100 | QS 100 | QS 100 | QS 100 |
| Preservatives | QS | QS | QS | QS |
| Fragrance | QS | QS | QS | QS |
| Performance | | | | |
| In vitro SPF | 14 | 10 | 39 | 42 |
| In vivo SPF | 18 | NA | NA | 36 |
| In vitro SPF Index | 2.3 | 3.8 | 6.5 | 7.2 |
| In vivo SPF Index | 3.0 | NA | NA | 6.2 |
| Star Rating (UVA) | + | ++ | ++ | ++ |

NA: data is not available

The control formulation of Table I is typical of conventional sunscreen formulations. The control formulation incorporates 6 wt % organic UV-actives and achieves an SPF value of 14 yielding an SPF Index of 2.33 (14/6). Formulation 1 demonstrates that almost the same SPF value may be achieved at much lower UV-actives concentration (2.6) by including about 1% of a coated silica cosmetic powder (SPF Index 3.8). In Formulations 1 and 3, the indicated silica and talc are coated with a mixture of triethoxycaprylylsilane, aluminum myristate, and disodium stearoyl glutamate.

Formulation 2 demonstrates a 2.8-fold increase over the control SPF value yielded by 6 wt % organic UV-actives is achievable by including 11 wt % uncoated cosmetic powder. The SPF Index is increased to 6.5. By using coated cosmetic powders (Formulation 3), an increase from SPF 39 to SPF 42 is achieved and the organic UV-actives are decreased from 6 wt % to 5.8 wt % and the SPF Index is increased to 7.2.

### Example 2

TABLE II

| | Example | Example | Example | Example |
|---|---|---|---|---|
| Ingredient | 4 | 5 | 6 | 7 |
| UV-Actives | | | | |
| Octinoxate | 5.8 | 5.7 | 5.7 | 5.7 |
| Cosmetic Powder | | | | |
| Materials | | | | |
| Coated silica | 1.1 | 1.2 | 1.3 | 1.4 |
| Coated kaolin | 6.5 | 8.0 | 10.0 | 12.0 |
| Remaining | | | | |
| Ingredients | | | | |
| Hydroxyethyl | 5.0 | 5.0 | 5.0 | 5.0 |
| Acrylate/Sodium | | | | |
| Acryloyldimethyl | | | | |
| Taurate opolymer | | | | |
| (and) Squalane | | | | |
| (and) Polysor- | | | | |
| bate 60 | | | | |
| (Simulgel ™ NS) | | | | |
| Deionized water | QS 100 | QS 100 | QS 100 | QS 100 |
| Preservatives | QS | QS | QS | QS |
| Fragrance | QS | QS | QS | QS |
| Performance | | | | |
| In vitro SPF | 20 | 21 | 23 | 23 |
| In vitro SPF | 3.4 | 3.7 | 4.0 | 4.0 |
| Index | | | | |
| Star Rating | + | + | + | + |
| (UVA) | | | | |
| Texture on skin | Trans- | Trans- | Trans- | Trans- |
| | parent | parent | parent | parent |

Procedure for Example 2: Oxybenzone was dissolved into Octinoxate by heating to approx. 70° C. Uncoated silica and uncoated talc were dispersed into the resulting Octinoxate solution and Hydroxyethyl Acrylate/Sodium Acryloyldimethyl Taurate copolymer (and) Squalane (and) Polysorbate 60 (Simulgel™ NS) was added. Water heated to about 70° C. was added to the powder suspended oil phase and emulsified by homogenization. Preservatives and fragrance are added to it as needed.

Example Formulations 4 through 7 maintain constant concentrations of organic UV-actives and coated silica while providing increasing concentrations of coated kaolin substrate. Table II shows that those conditions cause the SPF Index to increase to a plateau. The silica and kaolin of Formulations 4-7 are coated with a mixture of triethoxycaprylylsilane, aluminum myristate, and disodium stearoyl glutamate.

### Example 3

TABLE III

| | Exam- | Exam- | Exam- | Exam- | Exam- |
|---|---|---|---|---|---|
| Ingredient | ple 8 | ple 9 | ple 10 | ple 11 | ple 12 |
| UV-Actives | | | | | |
| Octinoxate | 2.75 | 2.60 | 2.85 | 2.40 | 2.90 |
| Oxybenzone | 2.75 | 2.60 | 2.85 | 2.40 | 2.90 |
| Cosmetic Powder | | | | | |
| Materials | | | | | |
| Coated silica | 1.2 | 1.2 | 1.1 | 1.1 | 1.3 |
| Coated Beadyl ™ | 9.4 | | | | |
| Beads (aluminum | | | | | |
| calcium sodium | | | | | |
| silicate) | | | | | |
| Coated kaolin | — | 9.0 | — | — | — |
| Coated mica | — | — | 9.8 | — | — |
| Coated titanated | — | — | — | 8.2 | — |
| mica, (Flamenco ™ | | | | | |
| Velvet) | | | | | |
| Coated talc | — | — | — | — | 10.0 |
| Remaining | | | | | |
| Ingredients | | | | | |
| hydroxyethyl | 4.8 | 4.8 | 4.8 | 4.8 | 4.8 |
| acrylate/sodium | | | | | |
| acryloyldimethyl | | | | | |
| taurate opolymer | | | | | |
| (and) squalane | | | | | |
| (and) polysor- | | | | | |
| bate 60 | | | | | |
| (Simulgel ™ NS) | | | | | |
| Deionized water | QS 100 | QS 100 | QS 100 | QS 100 | QS 100 |
| Preservatives | QS | QS | QS | QS | QS |
| Fragrance | QS | QS | QS | QS | QS |
| Performance | | | | | |
| In vitro SPF | 38 | 42 | 42 | 56 | 42 |
| In vitro SPF Index | 6.9 | 8.1 | 7.4 | 11.7 | 7.2 |
| Star Rating (UVA) | ++ | ++ | ++ | ++ | ++ |
| Texture on skin | Trans- | Trans- | Trans- | Trans- | Trans- |
| | parent | parent | parent | parent | parent |

In Table III, “silicate” refers to an aluminum calcium sodium silicate and the various cosmetic powder materials were coated with a mixture of triethoxycaprylylsilane, aluminum myristate, and disodium stearoyl glutamate. Flamenco Velvet refers to titanated mica similarly coated with a mixture of triethoxycaprylylsilane, aluminum myristate, and disodium stearoyl glutamate. In the various formulations, the concentration of UV-actives was maintained at 5.4±0.4%, the concentration of cosmetic powder was maintained at 10.6±0.6% (1.2±0.1% silica+9.4±0.6% other cosmetic powder). The data of Table III show that under these conditions, the SPF, and therefore the SPF Index, strongly depends on the nature of the specific silicate substrate.

### Example 4

TABLE IV

| | Example | Example |
|---|---|---|
| Ingredient | 13 | 14 |
| UV-Actives | | |
| Octinoxate | 2.06 | 1.64 |
| Oxybenzone | 2.06 | 0.82 |
| Avobenzone | — | 1.64 |
| Methyl | 2.06 | — |
| anthranilate | | |
| Cosmetic Powder | | |
| Materials | | |
| Coated silica | 1.3 | 1.1 |
| Coated kaolin | 9.6 | 7.7 |
| Remaining | | |
| Ingredients | | |
| Hydroxyethyl | 4.8 | 4.1 |
| Acrylate/Sodium | | |
| Acryloyldimethyl | | |
| Taurate opolymer | | |
| (and) Squalane (and) | | |
| Polysorbate 60 | | |
| (Simulgel ™ NS) | | |
| Deionized water | QS 100 | QS 100 |
| Disodium EDTA | — | 0.1 |
| Preservatives | QS | QS |
| Fragrance | QS | QS |
| Performance | | |
| In vitro SPF | 48 | 66 |
| In vitro SPF Index | 7.7 | 16.1 |
| Star Rating (UVA) | ++++ | ++++ |
| Texture on skin | Transparent | Transparent |

Formulations 13 and 14 show that the SPF Index may be controlled by varying the specific organic UV-active and the specific cosmetic powder substrate.

### Example 5

Surface treated, dried powder with coated UV absorbing oil was dispersed into “isopropyl myristate” and was followed by adding Simugel NS and Abil WE-09. Then a previously prepared water phase was added to the oil phase while homogenizing at approx. 4500 rpm.

TABLE V

| | Example |
|---|---|
| Ingredient | 15 |
| UV-Actives | |
| Octinoxate (Parsol MCX) | 2.25 |
| Octocrylene (Parsol 340) | 2.25 |
| Avobenzone (Parsol 1789) | 1.50 |
| Phase A (Aqueous) | |
| Deionized water | Balance to 100.00 |
| 1,3-Butylene Glycol | 2.00 |
| Sodium Chloride | 0.50 |
| Disodium EDTA | 0.10 |
| Phase B (Oil) | |
| Isopprpyl Myristate (IPM) | 17.00 |
| Polyglyceryl-4 | 5.00 |
| Isostearate (and) | |
| Cetyl PEG/PPG-10/1 | |
| Dimethicone (and) | |
| Hexyl Laurate | |
| (ABIL WE09) | |
| Hydroxyethyl | 3.00 |
| Acrylate/Sodium | |
| Acryloyldimethyl | |
| Taurate opolymer | |
| (and) Squalane | |
| (and) Polysorbate 60 | |
| (Simulgel ™ NS) | |
| Triethoxycaprylylsilane | 19.0 |
| (and) Aluminum Myristate | |
| (and) Disodium Stearoyl | |
| Glutamate (and) Kaolin | |
| (and) Mica (and) | |
| Tianium Dioxide*¹ | |
| Performance | |
| in vitro SPF | 50 |
| in vitro PFA | 15 |
| in vitro UVA/UVB ratio | 0.67 |
| in vitro Star Rating | +++ |
| in vitro SPF Index | 8.33 |
| in vivo SPF | 33 |
| in vivo PFA | 15 |

*¹Flamenco Velvet comprising a surface treatment of Triethoxycaprylylsilane (and) Aluminum Myristate (and) Disodium Stearoyl Glutamate provided onto a mixture of kaolin and titanated mica.

Formulation 15 is an example of a water-in-oil sunscreen prepared from an oven-dried wetcake. The overall treatment process and subsequent drying of the product is as described above. Phases A and B are prepared separately and then combined by homogenization. The final product is an oil-based, pourable sunscreen. In this system, the treated, dried powder is within the cosmetic oil (isopropyl myristate, “IPM”) and the sunscreen active oils remain with the treated powder due to the lipophilic nature of both the treatment and the actives. The system is stabilized with ABIL WE09 emulsifying agent.

### Example 6

Following examples are daily-use, “tinted sunscreens.”

TABLE VI

| | Comparative | Example | Example | Example |
|---|---|---|---|---|
| Ingredient | Example 2 | 16 | 17 | 18 |
| UV-Actives | | | | |
| Octinoxate | 0.72 | 0.72 | 0.67 | 1.56 |
| Octocrylene | 0.72 | 0.72 | 0.67 | 0.64 |
| Avobenzone | 0.72 | 0.72 | 0.23 | — |
| Cosmetic Powder | | | | |
| Materials | | | | |
| Coated titanated | — | 0.50 | 0.47 | 0.47 |
| mica, (Flamenco | | | | |
| Velvet) | | | | |
| Coated kaolin | — | 21.2 | 19.5 | 19.5 |
| Coated titanium | — | — | — | 0.63 |
| doxide | | | | |
| Coated yellow | — | — | 0.70 | 0.23 |
| iron oxide | | | | |
| pigment | | | | |
| Coated red | — | — | 0.20 | 0.05 |
| iron oxide | | | | |
| pigment | | | | |
| Coated black | — | — | 0.08 | 0.025 |
| iron oxide | | | | |
| pigment | | | | |
| Remaining | | | | |
| Ingredients | | | | |
| Hydroxyethyl | 3.2 | 3.2 | 2.4 | 3.2 |
| Acrylate/Sodium | | | | |
| Acryloyldimethyl | | | | |
| Taurate opolymer | | | | |
| (and) Squalane | | | | |
| (and) Polysor- | | | | |
| bate 60 | | | | |
| (Simulgel ™ NS) | | | | |
| Isopropyl | 3.0 | 3.0 | 3.0 | 3.0 |
| Myristate | | | | |
| Deionized water | QS 100 | QS 100 | QS 100 | QS 100 |
| Disodium EDTA | 0.1 | 0.1 | 0.1 | — |
| Preservatives | QS | QS | QS | QS |
| Fragrance | QS | QS | QS | QS |
| Performance | | | | |
| In vitro SPF | 8 | >50 | >50 | 35 |
| In vitro SPF | 3.7 | >20 | >30 | 15.9 |
| Index | | | | |
| Star Rating | +++ | +++ | +++ | ++ |
| (UVA) | | | | |
| Texture on | | Trans- | Trans- | Trans- |
| skin | | parent | parent | parent |
| | | | (colored) | (colored) |

Formulations 16, 17 and 18 are examples of oil-in-water sunscreens that provide both UVA and UVB protection. Each formulation was prepared by emulsifying the respective wetcake as described. In Example 16, avobenzone comprises the UVA filter. The Comparative Example contains the same amount of actives as Example 16, but the Comparative Example contains no cosmetic powder. This clearly demonstrates that the use of cosmetic powder in a given formulation greatly enhances the overall SPF. It should be noted that because the value is a ratio, the UVA Star Rating (+++) is the same for both the Comparative sample and Example 16. A much lower UVA value combined with a much lower overall SPF value may give the same UVA Star Rating (ratio) as is given by a much greater UVA value with a much larger overall SPF value. For example, 5:7 is the same ratio as 50:70 although the latter values are an order of magnitude larger. In example 17, the UVA protection is enhanced by employing the small primary particle size of the coated color pigments. Thus, the amount of avobenzone can be greatly reduced. It should be noted that example 17 is a tinted product with a reddish brown color.

Formulation 18 is free of Avobenzone. All UVA protection is derived from the primary particle size of the pigments. It is important to note that Examples 8-12, and 15 also have a ++ UVA rating. However, the average UVA ratio for these samples is ˜0.37. For Formulation 18 the UVA ratio is 0.50. This underscores the fact that the pigments are providing additional UVA protection. The above final product is colored with a “skin tone” tint.

In an embodiment, a silica-based cosmetic powder is surface treated by covalently boding an organic silane. In an embodiment, the organic silane is triethoxycaprylylsilane. In an embodiment, the surface-treatment agent is a complex of triethoxycaprylylsilane, aluminum myristate, and disodium stearoyl glutamate.

Example 19 is an example of a sunscreen in which the surface-treatment consists of covalently binding triethoxycaprylylsilane to kaolin (silica).

TABLE VII
Triethoxycaprylylsilane-bound “Octinoxate + Octocrylene +
Avobenzxone” W/O emulsion system

| | Comparative | |
|---|---|---|
| | Example 3 | Example 19 |
| Phase A | | |
| Water | 63.00 | 47.40 |
| 1,3 Butylene Glycol | 2.00 | 2.00 |
| Sodium Chloride | 0.50 | 0.50 |
| Disodium EDTA | 0.10 | 0.10 |
| Phase B | | |
| Kaolin (and) | — | 19.00 |
| Triethoxycaprylylsilane | | |
| Octinoxate [Parsol | 2.25 | 2.25 |
| MCX] | | |
| Octocrylene [Parsol | 2.25 | 2.25 |
| 340] | | |
| Avobenzone [Parsol | 1.50 | 1.50 |
| 1789] | | |
| Isopropyl Myristate | 20.40 | 17.00 |
| Polyglyceryl-4 | 5.00 | 5.00 |
| Isostearate (and) | | |
| Cetyl PEG/PPG- | | |
| 10/1 Dimethicone | | |
| (and) Hexyl Laurate | | |
| [ABIL WE09] | | |
| Hydroxyethylacrylate/ | 3.00 | 3.00 |
| Sodium Axryloylmethyl- | | |
| taurate Copolymer | | |
| (and) Squalane | | |
| (and) Polysorbate 60 | | |
| [SIMULGEL NS] | | |
| Total | 100.00 | 100.00 |
| in vitro SPF | 15 | 30 |
| in vitro PFA | 10 | 13 |
| in vitro UVA/UVB ratio | 0.63 | 0.68 |
| in vitro Star Rating | +++ | +++ |
| in vitro SPF Index | 2.5 | 5.0 |

Comparative Example 3, Table VII. Avobenzone was dissolved into the mixture of Octinoxate, Octocrylene, and isopropyl myristate with a propeller mixer with heating to approximately 75° C. Simulgel NS and WE-09 were added to the dissolved UV absorbents and maintained at approximately 65° C. until use. Sodium chloride, disodium EDTA and butylene glycol were dissolved in water by mild heating to approximately 50° C. in a separate vessel. The water phase was added to the oil phase while homogenizing at 4500 rpm. After a few minutes mixing, the component was cooled to ambient temperature. Then, SPF performance was determined using an SPF analyzer.

Example 19, Table VII. Triethoxycaprylylsilane treated, dried kaolin was prepared with high-speed mixer such as Henschel mixer according to procedures disclosed in U.S. Pat. No. 5,968,531. The treated kaolin was dispersed into an oil phase as described above for “Comparative Example 3.” A similarly-prepared water phase was added to the oil phase while homogenizing at 4500 rpm. After a few minutes mixing, the component was cooled down to ambient temperature. Then, SPF performance was examined with an SPF analyzer.

In an embodiment, an untreated cosmetic powder is dispersed into an oil phase as described above for Comparative Example 3. A water phase, prepared as above, is added to the oil phase with homogenization, as above. We have determined that untreated cosmetic powders, treated according to the present invention also enhance SPF performance.

What is claimed:

1. A wetcake method of making a surface-treated, cosmetic powder formulation comprising:
providing a cosmetic powder having at least one hydroxyl on a surface thereof;
contacting said cosmetic powder with an amount of a solvent to form a slurry;
contacting said slurry with a surface-treatment agent;
contacting said slurry with a polyvalent metal salt, wherein said polyvalent metal forms a metal-bridged, covalent complex with said cosmetic powder and said surface-treatment agent; and,
contacting said metal-bridged complex with a UV-active organic material.

2. The method of claim 1, further comprising separating a wetcake from said solvent.

3. The method claim 1, further comprising at least partially drying said wetcake.

4. The method claim 1, further comprising emulsifying said wetcake.

5. The method claim 1, wherein said formulation has an SPF Index of at least 3.0, at least 4.0, at least 6.0, at least 8.0, or at least 10.0.

6. The method claim 1, wherein said formulation comprises cosmetic powder at from about 0.5 wt % to about 35 wt %

7. The method claim 1, wherein said cosmetic powder is selected from the group consisting of silicates, surface modified silicates, organic polymers, and mixtures thereof.

8. The method claim 7, wherein said silicate is selected from the group consisting of silica, aluminum calcium sodium silicate, talc, mica, sericite, kaolin, and mixtures thereof;
wherein said surface-modified silicate is selected from the group consisting of: silica coated with triethoxycaprylylsilane, aluminum myristate, and disodium stearoyl glutamate; talc coated with triethoxycaprylylsilane, aluminum myristate, and disodium stearoyl glutamate; kaolin coated with triethoxycaprylylsilane, aluminum myristate, and disodium stearoyl glutamate; aluminum calcium sodium silicate coated with triethoxycaprylylsilane, aluminum myristate, and disodium stearoyl glutamate; mica coated with triethoxycaprylylsilane, aluminum myristate, and disodium stearoyl glutamate; titanated micas coated with triethoxycaprylylsilane, aluminum myristate, and disodium stearoyl glutamate; and mixtures thereof; and
wherein said organic polymer is selected from the group consisting of nylon, polyethylene, polystyrene, polymethylmethacrylate (PMMA), wool powder, cellulose powder, silk powder, starch, and mixtures thereof.

9. The method claim 1, wherein said surface-active agent is a residue derived from a compound selected from the group consisting of fatty acids, alkyl ether carboxylic acids, acylamino acids, 2-pyrrolidinone-5-carboxylic acids, acid polyamides, alkyl ether phosphoric acids, amphoterics, silanes, polysilanes, tetrahydropyran-2-carboxylic acids, tetrahydrofuran-2-acetyl carboxylic acids, salts thereof, and mixtures thereof;
wherein at least one carboxyl group of said surface-active agent is optionally substituted by a sulfate group or a phosphate group.

10. The method claim 1, wherein said organic UV-active is selected from the group consisting of Octinoxate (ethylhexyl methoxycinnamate), Oxybenzone (benzophenone-3), menthyl anthranilate, octocrylene, homosalate, octisalate, avobenzone, p-aminobenzoic acid, 2-ethoxyethyl-p-methoxy cinnamate, diethanolamine-p-methoxy cinnamate, digalloyl trioleate, 2,2-dihydroxy-4-methoxybenzophenone, ethyl-4-bis-(hydroxypropyl)aminobenzoate, 2-ethylhexyl-2-cyano-3,3-diphenyl acrylate, ethylhexyl-p-methoxy cinnamate, 2-ethylhexyl salicylate, glyceryl aminobenzoate, 3 3,5-trimethylcyclohexyl salicylate, lawsone with dihydroxyacetone, methyl anthranilate, 2-hydroxy-4-methoxy benzophenone, amyl-p-dimethylamino benzoate, 2-ethylhexyl-p-dimethylamino benzoate, 2-phenylbenzimidazole-5-sulphonic acid, red petroleum, 2-hydroxy-4-methoxybenzophenone-5-sulphonic acid, triethanolamine salicylate, and mixtures thereof.

11. A knead-blend method of making a surface-treated, cosmetic powder formulation comprising:
providing a cosmetic powder having at least one hydroxyl on a surface thereof;
contacting said cosmetic powder with an amount of a solvent sufficient to moisten said cosmetic powder and less than an amount to form a slurry;
kneading said cosmetic powder with said solvent to form a knead-blend;
contacting said knead-blend with a surface-treatment agent;
contacting said knead-blend with a polyvalent metal salt, wherein said polyvalent metal forms a metal-bridged, covalent complex with said cosmetic powder and said surface-treatment agent; and
contacting said metal-bridged complex with a UV-active organic material.

12. The method claim 11, further comprising at least partially drying said knead-blend.

13. The method claim 11, further comprising emulsifying said knead-blend.

14. The method claim 11, wherein said formulation has an SPF Index of at least 3.0, at least 4.0, at least 6.0, at least 8.0, or at least 10.0.

15. The method claim 11, wherein said formulation comprises cosmetic powder at from about 0.5 wt % to about 35 wt %

16. The method claim 11, wherein said cosmetic powder is selected from the group consisting of silicates, surface modified silicates, organic polymers, and mixtures thereof.

17. The method claim 16, wherein said silicate is selected from the group consisting of silica, aluminum calcium sodium silicate, talc, mica, sericite, kaolin, and mixtures thereof;
wherein said surface-modified silicate is selected from the group consisting of: silica coated with triethoxycaprylylsilane, aluminum myristate, and disodium stearoyl glutamate; talc coated with triethoxycaprylylsilane, aluminum myristate, and disodium stearoyl glutamate; kaolin coated with triethoxycaprylylsilane, aluminum myristate, and disodium stearoyl glutamate; aluminum calcium sodium silicate coated with triethoxycaprylylsilane, aluminum myristate, and disodium stearoyl glutamate; mica coated with triethoxycaprylylsilane, aluminum myristate, and disodium stearoyl glutamate; titanated micas coated with triethoxycaprylylsilane, aluminum myristate, and disodium stearoyl glutamate; and mixtures thereof; and
wherein said organic polymer is selected from the group consisting of nylon, polyethylene, polystyrene, polymethylmethacrylate (PMMA), wool powder, cellulose powder, silk powder, starch, and mixtures thereof.

18. The method claim 11, wherein said surface-active agent is a residue derived from a compound selected from the group consisting of fatty acids, alkyl ether carboxylic acids, acylamino acids, 2-pyrrolidinone-5-carboxylic acids, acid polyamides, alkyl ether phosphoric acids, amphoterics, silanes, polysilanes, tetrahydropyran-2-carboxylic acids, tetrahydrofuran-2-acetyl carboxylic acids, salts thereof, and mixtures thereof;
wherein at least one carboxyl group of said surface-active agent is optionally substituted by a sulfate group or a phosphate group.

19. The method claim 11, wherein said organic UV-active is selected from the group consisting of Octinoxate (ethylhexyl methoxycinnamate), Oxybenzone (benzophenone-3), menthyl anthranilate, octocrylene, homosalate, octisalate, avobenzone, p-aminobenzoic acid, 2-ethoxyethyl-p-methoxy cinnamate, diethanolamine-p-methoxy cinnamate, digalloyl trioleate, 2,2-dihydroxy-4-methoxybenzophenone, ethyl-4-bis-(hydroxypropyl)aminobenzoate, 2-ethylhexyl-2-cyano-3,3-diphenyl acrylate, ethylhexyl-p-methoxy cinnamate, 2-ethylhexyl salicylate, glyceryl aminobenzoate, 3 3,5-trimethylcyclohexyl salicylate, lawsone with dihydroxyacetone, methyl anthranilate, 2-hydroxy-4-methoxy benzophenone, amyl-p-dimethylamino benzoate, 2-ethylhexyl-p-dimethylamino benzoate, 2-phenylbenzimidazole-5-sulphonic acid, red petroleum, 2-hydroxy-4-methoxybenzophenone-5-sulphonic acid, triethanolamine salicylate, and mixtures thereof.
